(19) United States
(12) Patent Application Publication
(10) Pub. No.: US 2015/0140100 A1
(43) Pub. Date: May 21, 2015

(54) MEDICAL DEVICE FOR TREATMENT OF WOUNDS

(71) Applicants: Lars-Magnus Bjursten, (US); Sven-Erik Nilsson, Skäret (SE); Migrata U.K. Limited, Limassol (CY)

(72) Inventors: Lars-Magnus Bjursten, Limhamn (SE); Sven-Erik Nilsson, Skaret (SE)

(21) Appl. No.: 14/405,558

(22) PCT Filed: May 29, 2013
(86) PCT No.: PCT/SE2013/050619

§ 371 Date: Dec. 4, 2014

Related U.S. Application Data
(60) Provisional application No. 61/655,040, filed on Jun. 4, 2012.

(30) Foreign Application Priority Data
Jun. 4, 2012 (SE) 1250581-4

(51) Int. Cl. A61L 15/44; A61L 15/18; A61L 15/42

(52) U.S. Cl. CPC A61L 15/44; A61L 15/425; A61L 15/18; A61L 2300/102; A61L 2300/404; A61L 2300/41; A61L 2300/602
USPC 424/489; 424/617

(57) ABSTRACT

The present invention describes a medical device comprising a set of particles of titanium oxide, wherein at least a substantial amount of the particles are of micrometer—millimeter size, and wherein at least 10 wt % of the titanium oxide is in the amorphous form.

### FIELD OF THE INVENTION

The present invention relates to medical devices for treatment of wounds, especially chronic wounds.

### TECHNICAL BACKGROUND

Chronic wounds are a complication seen in many patients. For example, diabetes patients commonly suffer from chronic wounds due to insufficient blood circulation and many elderly or severely ill patients suffer from bed sores/pressure sores due to a constant pressure to the limbs when lying down for long periods of time.

The primary therapy of chronic wounds is of course to treat the underlying conditions causing the wound. However, other direct treatment of the wound is also important.

Burns, leg ulcers, diabetic foot ulcers and bed sores are all often more or less infected with bacteria. This is a complication, which may lead to amputation or even death in the case of the infection evolving to sepsis. To avoid this, systemic antibiotic treatment is widely used in connection with the treatment of such wounds, which as a side effect contributes to the increasing antibiotic resistance in bacteria. Therefore, several antibacterial wound dressings have been developed for replacing or assisting therapy with systemic antibiotics.

The bacteriostatic and fungistatic effect of silver is well known and silver has been used clinically for many years.

WO02078755 relates to a medical dressing comprising a complex of silver which is said to be capable of releasing antimicrobial silver ion activity to a wound. The medical dressing disclosed in WO02078755 comprises a silver compound and is capable of releasing antimicrobial silver activity in the range of 50-10000 μg per cm² dressing to a wound. At the same time, the dressing is said to be capable of absorbing more than 0.09 g per cm² dressing of wound exudates. The dressing disclosed in WO02078755 comprises the silver compound in the form of silver ions in the form of a complex stabilising the silver against reduction to free silver.

However, several recent studies report of bacteria having developed resistance against silver. Thus, there is a need for novel wound dressings without silver. In addition, silver used in various products has been shown to have negative effects on the environment, especially when not properly disposed of.

One aim of the present invention is to provide a medical device being effective for treating wounds and which overcomes the above stated problems.

### SUMMARY OF THE INVENTION

The stated purpose above is achieved by a medical device comprising a set of particles of titanium oxide, wherein at least a substantial amount of the particles are of micrometer—millimeter size, and wherein at least 10 wt % of the titanium oxide is in the amorphous form.

The present invention is directed to providing a medical device directed to optimal structure, i.e. in the shape of particles, grains or granules, as well as material, i.e. titanium oxide, where at least 10 wt % is in the amorphous form. As the amorphous form of titanium oxide is not catalytic active, which is the case for the crystalline forms, the amorphous form provides an antibacterial effect. This is further explained below.

When titanium is exposed to air or water, an oxide layer is spontaneously formed. This spontaneously formed oxide layer is 4-10 nm thick and consists predominantly of TiO₂, Ti(IV), with smaller amounts of Ti(III) and Ti(II) present in the oxide, The anti-inflammatory and antibacterial effects of titanium are based on the chemical properties of TiO₂ at its surface and may work in several different ways, all related to the exposed surface area. As previously shown (reference 2), TiO₂ has the ability to directly scavenge ROS (reactive oxygen species). One possible mechanism is through a set of catalytic redox reactions that has been suggested for the breakdown of hydrogen peroxide, superoxide and peroxynitrite on titanium dioxide surfaces:

2TiO₂+2O₂⁻+2H⁺→Ti₂O₃+2O₂+H₂O (1a)

2TiO₂+H₂O₂→Ti₂O₃+O₂+H₂O (1b)

Ti₂O₃+OONO⁻→2TiO₂+NO₂⁻ (2a)

Ti₂O₃+H₂O₂→2TiO₂+H₂O (2b)

Of special interest with respect to the antibacterial effects of titanium is the possibility that TiO₂ may also react directly with H₂O₂ and form a Ti-peroxy gel, TiOOH(H₂O)ₙ, on the oxide surface. ESR (electron spin resonance) measurements have also shown that superoxide radicals are present in the Ti-peroxy gel, indicating either trapping of superoxide in the gel or direct reaction between superoxide and Ti(IV) in the Ti-peroxy gel. Complexes similar to the Ti-peroxy gel might also be formed between TiO₂ and peroxynitrite. It was recently shown that peroxynitrous acid, the protonated form of peroxynitrite (pKa=6.8), forms a complex similar to the Ti-peroxy gel with Ti(IV) under acidic conditions. Moreover, the blue tint sometimes found in tissue surrounding titanium implants suggests that Ti(IV) reacts with ROS and forms stable Ti(III) complexes. It has also been shown that the thickness of the titanium oxide layer on implants increases with time in vivo, suggesting that Ti metal might act as a sink for oxygen species. All of these reactions might be involved in the direct breakdown of ROS that occurs on the TiO₂ surface and the linked anti-inflammatory effect.

Titanium (that is titanium metal with a surface layer of titanium oxide) has been reported to reduce inflammation (Overgaard, Danielsen et al. 1998) and also to be less susceptible to infections than other materials (Johansson, Lindgren et al. 1999). There are also reports describing unique properties of titanium due to its chemical interactions with reactive oxygen species (ROS). The catalytic property of titanium has been shown to be related to the titanium oxide on the surface being present on surfaces composed of only titanium oxide (Sahlin 2006 et al). Such a catalytic property is e.g. described in the U.S. patent application no. 2005074602 to Bjursten et al and also in the generation of titanium peroxy compounds (Tengvall, Elwing et al. 1989; Tengvall, Lundstrom et al. 1989) with anti-inflammatory (Larsson, Persson et al. 2004) and bactericidal properties (Tengvall, Hornsten et al. 1990). The above beneficial properties of titanium seems thus to be linked to its chemical interaction with a living tissue environment. The formation of titanium-peroxy compounds seems not to be linked to the catalytic properties of titanium dioxide but as an alternative reaction to the catalytic breakdown of oxygen radicals. The catalytic reactions are favoured by the presence of crystalline phases of the titanium dioxide. Thus, for the present invention the non-crystalline form of the titanium oxide, the amorphous phase, is preferred.

As hinted above, the present invention is directed to providing a set of particles exhibiting a high level of the amorphous form of titanium oxide, or titanium dioxide, to achieve an increased anti-bacterial effect. As such, the medical device according to the present invention is effective in control of infectious diseases.

Medical usages of particles of titanium, titanium alloy or titanium oxide are known today. For instance, it has long been known that titanium, its oxides and alloys are biocompatible and hence are used in various medical applications. For example, operations and wounds in the body often bring about inflammation and/or infections, which is the case also in connection with implantations, especially in connection with bone tissue, e.g. hip joints and dental applications. In e.g. WO 2008/103082 there is disclosed particles of microstructure comprising titanium, titanium alloy, at least one titanium oxide or a combination thereof and their use in medical applications. The disclosed particles have a surface with at least a substantial part consisting of at least one type of titanium oxide. The particles are brought into contact with at least one infected site in a human or animal body by insertion, injection or implantation. The infected site exhibits the inflammatory and/or bacterial condition. Furthermore, WO2008/103082 refers to an injectable suspension comprising the particles and a fluid vehicle for use as a medicament. Examples of conditions being treated with the injectable suspension are periodontitis, periimplantitis, and osteitis. Due to the fact of the small size of the particles, these could easily be brought into contact with an infected site present in the human or animal body. Specific examples are infected sites in the mouth or close to the teeth, that is for dental applications, but also e.g. in the intestine or other organs or tissues. An important example is bone tissue. In addition to being injected into inflamed and/or infected tissue, the particles of microstructure or the injectable suspension disclosed in WO 2008/103082 may also be injected into or inserted into non-inflamed and/or non-infected sites of a human or animal body, e.g. the intestine, liver, spleen, pancreas or the kidneys. One example of use of the particles of microstructure or the injectable suspension are as carriers of medicaments to specific parts of the human or animal body, where the particles either work just as a carriers or as active medicaments in combination with the other medicaments at the site intended to be contacted.

Furthermore, in US2008/0317830A1 a topical formulation for the treatment of wounds is disclosed. The disclosed topical formulation comprises 1-30% (by weight) particles of for example titanium dioxide together with a pharmaceutically acceptable petrolatum base. The particles are about 100 μm in diameter. The pharmaceutically acceptable base may also comprise for example mineral oils and vegetable oils. The formulation disclosed in US2008/0317830A1 may further comprise one or more active agents such as antibacterial agents, anti fungal agents, topical steroids, topical anesthetics and anti-inflammatory agents.

Neither the topical formulation according to US2008/0317830A1 not the injectable suspension according to WO2008/103082 are directed to providing a set of particles having a high level of titanium oxide in the amorphous form, such as the present invention.

### SPECIFIC EMBODIMENTS OF THE INVENTION

Below, specific embodiments of the present invention are disclosed.

According to one specific embodiment of the present invention, the particles are made of titanium dioxide.

As may be understood from above, there are different forms of titanium oxides. The normally mentioned titanium oxide forms are titanium(II) oxide (titanium monoxide, TiO), which is a non-stoichiometric oxide, titanium(III) oxide (dititanium trioxide, Ti₂O₃), trititanium pentaoxide (Ti₃O₅) and titanium-(IV) oxide (titanium dioxide, TiO₂). However, there exists also other oxides, such as a composition between TiO₂ and Ti₃O₅, and they have the general formula TiₙO₂ₙ₋₁ where n ranges from 4-9. Worth mentioning, titanium(II) oxide (TiO) can be prepared from titanium dioxide and titanium metal at a temperature of 1500° C. and titanium(III) oxide can be prepared by reacting titanium dioxide with titanium metal at a temperature of 1600° C. However, titanium(IV) oxide or titanium dioxide (titanium dioxide in the following) is the naturally occurring oxide of titanium and one could say that it is the most important oxide form of titanium. When used as a pigment, it is called titanium white, due to its whitish appearance. Titanium dioxide occurs in nature as the naturally occurring crystalline forms of rutile, anatase and brookite, of which rutile is the most stable form.

In accordance with what is mentioned above, the particles according to the present invention comprise titanium oxide which preferably is in the form of titanium dioxide. A high level of the amorphous form of titanium dioxide should be promoted according to the present invention as this provides increased anti-bacterial effect.

The result of oxidation of titanium is temperature dependent. To obtain the amorphous form the oxidation should be performed at low temperature, for instance below 300° C. For instance, hydrothermal crystallization of amorphous titanium dioxide at 300-600° C. follows the reaction path of: amorphous reactant to anatase to rutile. The amorphous titanium oxide is formed spontaneously at atmospheric conditions but may be accelerated and made thicker by a careful heating in the presence of oxygen, e.g. in the form of air.

As hinted above, the material of the particles is essential in relation to the present invention. Titanium oxide, preferably titanium dioxide, in amorphous form is present according to the present invention. It should, however, be noted that also other particles may be admixed in the set of particles. For instance, some particles may be made of titanium metal or alloy, or having such a core and also comprising an oxidized shell. Therefore, for instance a set of particles where some particles are of pure titanium dioxide, but other of titanium metal is fully possible according to the present invention. However, at least 10 wt % amorphous titanium oxide is always present.

Moreover, the geometrical structure and size of the particles are also an important feature. According to the present invention, the particles are of micrometer—millimeter size. In relation to the present invention, this implies that the particles have a “diameter” in the range of 10 μm-10 mm, such as in the range of 10 μm-5 mm. The expression “diameter”, as discussed below, should be seen as an average measure of the distance from one side of the particle to the other through a geometrical centre, and should not be related to implying a measure for only round shapes. The particles according to the present invention are not perfect spheres as discussed below.

The set of particles according to the present invention is intended to exhibit a capillary action. This may be accomplished in different ways according to the present invention. The particles may for instance be very small, such as in the range of 10 μm-0.1 mm, and as such have a large specific surface area (m²/g). The capillary action is in such case obtained between different particles. However, this may also be accomplished by porosity. In such a case, the particles may instead have sizes up to 5 mm, or at least 2 mm, and have pores which as such have an attracting action on wound liquid/pus. In such cases the particles may be addressed as grains or granules. The pores of such granules may be so called continuous pores going through the particles from one side to the other side, implying at least two openings on the surface of the particle. The pores may also resemble caves with only one opening on the particle surface. These caves may also be pores going deep inside of the particle but not through the entire structure. The caves may be of different length, stretching from one side of the particle to the other side of the same particle or appear as holes on the surface of the particles. The cavities may have an irregular shape and be that of a channel or hole inside the particle. Furthermore, there may also be provided cavities which are nearer the surface and not as deep.

The capillary action of the set of particles according to the present invention is of interest as this ensures an attracting force on wound liquid/pus.

Moreover, the entire structure of the particles or granules may have an irregular shape implying that the surface is wavy or also having a geometrical shape not being a sphere, such as having an oval cross section or the like. Possible shapes are e.g. spikes, flakes, chips or similar or combinations thereof. Furthermore, structures incorporating all of the above features, such being almost oval, having continuous pores and an irregular surface and so on, are of course totally possible according to the present invention. As discussed above, promoting an anti-bacterial effect is a focus according to the present invention. Therefore, according to one specific embodiment of the present invention, at least 50 wt % of the titanium oxide is in the amorphous form. This specification implies a higher “purity” degree in the entire set of particles. According to another specific embodiment, at least 75 wt % of the titanium oxide is in the amorphous form, such as at least 95 wt % of the titanium oxide is in the amorphous form. As one should understand, these higher levels of amorphous titanium dioxide according to the present invention is linked to producing oxides where the formation of the crystalline forms are depressed .

The set of particles according to the present invention may be incorporated in different forms of medical devices. According to one specific embodiment of the present invention, the medical device also comprises a bandage holding the particles on a surface of the bandage.

The expression “bandage” should be seen as any form of medical device where the set of particles can be safely uphold, such as for instance sticking plasters or membranes, etc.

To combine wound dressings with membranes or such have been made before. For instance, US2009/0209897A1 discloses a photoactivated antimicrobial wound dressing comprising a photocatalytic membrane. The photocatalytic membrane comprises a bacterial cellulose hydrogel membrane having photocatalytic particles immobilized within the membrane. The photocatalytic particles are activated when exposed to light, at which time they react with oxygen-based species forming reactive oxygen species. The reactive oxygen species further react with microbes to kill the microbes. The bacterial cellulose hydrogel membrane may be prepared from cellulose-producing bacteria and the photocatalytic particles may be titanium dioxide nanoparticles having a particle size ranging from about 5 nm to about 100 nm. The photocatalytic membrane is activated by ultraviolet light having a wavelength of about 365 nm. Moreover, US2010/0274176A1 discloses wound care systems. The wound care systems may include a first material comprising one or more fibers or porous media. The one or more fibers or porous media may be coated with a second material that, upon exposure to light, is capable of inhibiting the growth of bacteria and killing the bacteria to render the wound care system sterile and/or is capable of increasing the absorbency of the first material. The first material may be cotton, or any suitable fibrous material, the second material may be TiO2, and the light may be UV or visible light. The anti-bacterial article may further comprise a programmable switching circuit coupled to the light source.

It should, however, be noted that neither US2009/0209897A1 nor US2010/0274176A1 are related to a set of particles such as according to the present invention.

According to yet another specific embodiment of the present invention, the medical device also comprises a perforated bag containing the particles, which perforated bag allows for a liquid to penetrate.

EP1112046 discloses an implant prosthesis comprising a batch of mixture of porous grains or granular material of tissue-compatible type and disintegrated tissue-compatible biological material, preferably endogenous material such as bone meal. The grains or granular material is titanium, polymer or dextran. The size of the grains or granules is between 0.1 and 5 mm. The batch further comprises a tissue-biocompatible component which allows modeling or moulding of the batch. The batch is enclosed in a pouch or wrap made of a flexible tissue-compatible material having pores, apertures or perforations of a size which allows ingrowth and outgrowth of the biological material.

Also in this case it should be noted that the set of particles exhibiting a lowest level of amorphous form of titanium oxide according to the present invention are different from the grains or granules according to EP1112046. Moreover, the purpose of the present invention, relating to wounds is also different.

It should be noted that the set of particles according to the present invention may be presented in a dry form, which is positive when formulating. Therefore, according to one specific embodiment, the set of particles is a dry mixture of the particles. However, such a dry form may be of interest to combine with a wetting agent in a kit. A totally dry formulation in contact with a wound may promote scar formation. However, e.g. when contained for instance in a perforated bag, which allows for liquid penetration however not for particles falling out, the set of particles may be held in a dry formulation. However, also such medical device may be combined with at least one wetting agent in a kit.

The wetting agent may be any suitable type available today, for instance a gel, dispersing agent, protein, peptide, carbohydrate or an ampholyte or the like.

According to the present invention there is also provided the medical device for use, in the treatment of a wound. The medical device according to the present invention is brought into contact with the wound, e.g. the chronic wound, to treat. According to one specific embodiment, the wound is a chronic wound, such as chronic wounds caused by insufficient blood circulation of e.g. diabetic patients or caused by bed sores/pressures of elderly.

According to another embodiment there is provided use of a set of particles of titanium oxide, wherein at least a substantial amount of the particles are of micrometer—millimeter size, and wherein at least 10 wt % of the titanium oxide is in the amorphous form, for the treatment of a chronic wound. Moreover, the use may also be directed to a treatment of a wound by achieving an absorbing effect in the wound. This is related to the capillary effect of the set of particles or e.g. porous granules.

Preliminary Clinical Data on Topical Treatment with Titanium Oxide Particles in Wound Dressings

| | Time | | Area | Purulent |
|---|---|---|---|---|
| Patient | (weeks) | Treatment | (cm × cm) | discharge |
| Diabetes patient 1 | 0 | No/Start | 5 × 6 | ++++ |
| | 1 | AP | 5 × 4 | ++ |
| | 2 | AP | 4 × 3 | ++ |
| Diabetes patient 2 | 0 | No/Start | 5 × 4 | ++ |
| | 1 | CP | 4 × 4 | ++ |
| | 2 | CP | 3 × 4 | + |
| Venous insufficiency | 0 | No/Start | 4 × 4 | + |
| | 1 | CP | 3 × 4 | + |
| | 2 | CP | 3 × 3 | − |

The patients were treated with either amorphous (AP) or crystalline particles (CP) in wound dressings that were changed daily during 2 weeks and evaluated before and after one and two weeks of treatment. No antibiotics were administered during the treatment period.

As may be noted from the treatments, three different patients were treated, two having diabetes and one having venous insufficiency. The purulent discharge was evaluated from the start when no treatment was employed (week 0). Then the treatment was started an evaluated after one and two weeks, respectively. The purulent discharge is a measure of the level of inflammation/infection. The higher rating, i.e. more +, implies a higher level of purulent discharge and thus inflammation/infection.

As notable, both forms of titanium oxide particles seemed to promote healing of chronic wounds evaluated as the size of the wound. The amorphous particles seemed to be more effective in reducing clinical signs of infection and inflammation as evidenced by purulent discharge. This may be noted by AP having a faster and higher level of effect when suppressing the measure of purulent discharge (number of +).

1. A medical device comprising a set of particles of titanium oxide, wherein at least a substantial amount of the particles are of micrometer—millimeter size, and wherein at least 10 wt % of the titanium oxide is in the amorphous form.

2. The medical device according to claim 1, wherein the titanium oxide is titanium dioxide.

3. The medical device according to claim 1, wherein at least 50 wt % of the titanium oxide is in the amorphous form.

4. The medical device according to claim 1, wherein at least 75 wt % of the titanium oxide is in the amorphous form.

5. The medical device according to claim 1, wherein at least 95 wt % of the titanium oxide is in the amorphous form.

6. The medical device according to claim 1, which medical device also comprises a bandage holding the particles on a surface of the bandage.

7. The medical device according to claim 1, which medical device also comprises a perforated bag containing the particles, which perforated bag allows for a liquid to penetrate.

8. The medical device according to claim 1, wherein the set of particles is a dry mixture of the particles.

9. The medical device according to claim 1, which medical device is a kit also comprising at least one wetting agent.

10. The medical device according to claim 1, for use in the treatment of a wound.

11. The medical device according to claim 1, for use in the treatment of a chronic wound.

12. A method of treating a wound, comprising applying a medical device to the wound, the medical device comprising a set of particles of titanium oxide, wherein at least a substantial amount of the particles are of micrometer—millimeter size, and wherein at least 10 wt % of the titanium oxide is in the amorphous form.

13. The method of claim 12, wherein the medical device has an absorbing effect on the wound.

14. The method of claim 12, wherein the particles are made of titanium dioxide and wherein at least 50 wt % of the titanium oxide is in the amorphous form.

15. The method of claim 12, wherein at least 75 wt % of the titanium oxide is in the amorphous form.

16. The method of claim 12, wherein at least 95 wt % of the titanium oxide is in the amorphous form.
